(19) United States
(12) Patent Application Publication
(10) Pub. No.: US 2019/0076507 A1
(43) Pub. Date: Mar. 14, 2019

(54) Artificial Red Blood Cell Having Ability to Inhibit Conversion of Hemoglobin into Methemoglobin

(71) Applicant: Public University Corporation Nara Medical University, Nara (JP)

(72) Inventors: Hiromi Sakai, Nara (JP); Magohei Yamada, Nara (JP)

(21) Appl. No.: 16/079,918

(22) PCT Filed: Mar. 1, 2017
(86) PCT No.: PCT/JP2017/008187

§ 371 Date: Aug. 24, 2018

(30) Foreign Application Priority Data
Mar. 2, 2016 (JP) 2016-040494

(51) Int. Cl. A61K 38/42; A61K 38/43; A61K 47/16; A61K 47/24; A61K 47/28; A61K 47/34; A61K 9/127; A61P 7/08; A61K 38/17

(52) U.S. Cl. CPC A61K 38/42; A61K 38/43; A61K 47/16; A61K 47/24; A61K 38/1722; A61K 47/34; A61K 9/1271; A61P 7/08; A61K 47/28

(57) ABSTRACT

Disclosed is an artificial red blood cell containing purified and enriched hemoglobin that substantially has no enzymatic activity to reduce methemoglobin, transformation of the hemoglobin into methemoglobin. Provided is an artificial red blood cell which comprises: an aqueous solution that contains NADH and/or NADPH and hemoglobin; and a capsule that includes the aqueous solution, wherein the aqueous solution and the capsule substantially have no enzymatic activity to reduce methemoglobin.

### TECHNICAL FIELD

The present invention relates to an artificial red blood cell consisting of an aqueous solution and a capsule including the aqueous solution therein and to a method for producing the artificial red blood cell, wherein the aqueous solution is comprised of hemoglobin (Hb) and NADH and/or NADPH. More specifically, the present invention relates to an artificial red blood cell and a method for producing the artificial red blood cell having ability to inhibit conversion of Hb to methemoglobin (metHb).

### BACKGROUND

The current system for blood donation and transfusion has been established as an indispensable technique for clinical medicine, providing high level of safety. However, the risk of infection by transfusion is not completely eliminated. It also faces the threat of emerging infection. Accidents occur in which blood of a wrong blood type is administered to a patient due to medical malpractice. In addition, it is often difficult to conduct a blood type cross-matching test when an emergency transfusion is required. Storage life under refrigeration of red cell concentrate is three weeks in Japan and six weeks in the U.S. and Europe after blood donation. When the term expires, the red cell concentrate is forced to be discarded. As the storage life is short, it is likely that blood for transfusion cannot be sufficiently supplied, when the demand of blood for transfusion is increased, such as in a large-scale disaster or an emergency situation.

To overcome these problems, an artificial blood preparation has been developed (non-patent document 1). Hemoglobin is the most abundant protein contained in the blood. Hemoglobin is a protein which reversibly binds and dissociates the molecular oxygen. In short, the main function of the blood is oxygen transportation, and signifies the importance of the oxygen supply in maintenance of life. As it has been known for many decades that Hb binds and dissociates the oxygen, many of the substances developed as artificial oxygen carrier (artificial red blood cells) are based on the Hb. The so-called modified Hb includes (i) an intra-molecule cross-linked Hb in which Hb is prevented from dissociating into subunits, (ii) a polymerized Hb in which intermolecular cross-linking is carried out by glutaraldehyde, activated raffinose, or the like, in order to increase molecular weight of Hb; and (iii) a polymer-bound Hb in which polyethylene glycol, dextran, or albumin is chemically bound to Hb. As the modified Hb is relatively simple to produce, development of many of the modified Hb preparations has proceeded as far as clinical trials. However, development of almost all of them is disrupted because of their toxicity. The possible explanations includes as follows: that the Hb has a strong affinity or reactivity to nitric oxide (NO), a vascular endothelial relaxation factor, and inactivate NO, leading to vasoconstriction and peripheral circulation failure; that the blood vessel wall and the heart muscle are damaged because the blood vessel wall is directly exposed to a byproduct generated by the reaction of Hb with active oxygen or the like; and that Hb causes various side effects because the Hb is easily leaked out of the blood vessel due to the small particle size (non-patent document 2). In other words, the Hb is as toxic as the hemolysis where the Hb, which should be in the red blood cell, is out of the red blood cell.

The inventors of the present invention have been involved in the development of a Hb vesicle which contains Hb in a liposome (Hb-V). Hb-V is an artificial red blood cell which encapsulates highly-purified and highly-concentrated Hb (30-42 g/DL) in a liposome (non-patent document 3). The liposome is comprised of four components: 1,2-dipalmitoyl-sn-glycero-3-phosphatidylcholine (DPPC); cholesterol; 1,5-O-dihexadecyl-N-succinyl-L-glutamate (DHSG); and 1,2-distearoyl-sn-glycero-3-phosphatidylethanolamine-N-PEG₅₀₀₀(DSPE-PEG₅₀₀₀). The concentration of Hb in the Hb-V dispersion is 10 g/dL and the particle size is controlled to 250 to 280 nm. So far, studies in animal models have confirmed a high safety level. Experiments in animal models have shown the efficacy in resuscitation from hemorrhagic shock, high level of hemodilution, administration for anemia therapy, reduction of infarct region of cerebral infarction, oxygenation in the ischemic region, supplement for extracorporeal circulation circuit, an organ perfusion solution, a CO carrier, and others.

Meanwhile, one Hb molecule is composed of four subunits (α₂β₂) and each of the subunits has one heme, an oxygen-binding site. When the center iron, or, the iron in the center of the heme is divalent (ferrous, Fe²⁺), the iron binds reversibly the oxygen. The state in which oxygen is bound to the center iron in a divalent state is referred to as oxyhemoglobin (HbO₂), and a state in which oxygen is not bound is referred to as deoxyhemoglobin (deoxyHb). HbO₂ with bound oxygen is gradually converted by autoxidation to metHb whose iron is trivalent (ferric, Fe³⁺) and does not bind to oxygen. At the time, a superoxide anion (O₂⁻.) is released by a reaction of the following chemical formula

HbO₂→metHb+O₂⁻.

The O₂⁻. is converted to hydrogen peroxide by disproportionation reaction and promotes oxidation of HbO₂ and deoxyHb. Red blood cells have mechanisms to reduce metHb and to remove active oxygen to suppress these reactions. Known systems to reduce metHb are as follows.
(i) reducing agents such as ascorbic acid and glutathione directly react with metHb. Oxidized dehydroascorbic acid and oxidized glutathione are returned to the reduced species by an enzyme.
(ii) NADH-metHb reductase is reported as a NADH-based metHb reductase using nicotinamide adenine dinucleotide (NADH) as substrate. It was found that there is a mechanism that metHb is reduced with cytochrome b₅ as electron mediator and through the action of NADH-cytochrome b₅ reductase. Oxidized NAD⁺ is restored to NADH by the Embden-Meyerhof pathway. NADH cytochrome b₅ reductase is present on the red blood cell membrane as well as dissolved in the red blood cells.
(iii) MetHb is reduced by the action of NADPH metHb reductase with nicotinamide adenine dinucleotide phosphate (NADPH) as a substrate. The oxidized NADP⁺ is restored to NADPH by a pentose phosphate pathway.
(Iv) The red blood cells also contain a superoxide dismutase (SOD) which converts O₂⁻ into hydrogen peroxide (H₂O₂), and a catalase which eliminates hydrogen peroxide.

Hemoglobin as a raw material for producing the artificial oxygen carrier is purified and isolated from human red blood cells and red blood cells of domestic animals. A general purification method is as follows. First, blood is supplemented with anticoagulant and centrifuged to precipitate red blood cells. The plasma layer, platelets and the buffy coat (white blood cells) of the supernatant are removed, and the precipitated red blood cells are collected. The precipitated red blood cells are suspended in saline, dispersed and centrifuged. The supernatant is removed and the precipitated red blood cells are collected. The washed red blood cells can be obtained by repeating the above-mentioned steps for a few rounds. Hemoglobin is released with hemolysis by adding distilled water to the red blood cells. The Hb solution is referred to as hemolysate. Stroma (membrane of red blood cells) are removed by either of the following procedures: (i) removing stroma by treating with an ultrafiltration membrane having molecular weight cut-off of about 1000 kDa, and filtrating soluble substances; or (ii) removing stroma by ultrafiltration. The Hb solution obtained as above is referred to as stroma free hemoglobin (SFHb), which is mainly comprised of Hb but also contains water-soluble enzyme systems present in the red blood cells. SFHb is further filtrated with ultrafiltration membrane having molecular weight cut-off of 8 to 10 kDa and concentrated. The metHb reducing system can be restored by adding enzyme substrate to the SFHb which contains the enzyme system so that the above-mentioned conversion of Hb to metHb can be suppressed. In addition, when artificial red blood cells are produced by encapsulating crude hemoglobin (SFHb) supplemented with substrate for the enzyme system, the resulting artificial red blood cells can delay the conversion of Hb to metHb by metabolic turnover involving the metHb reducing enzyme system.

“Disadvantage” of use of SFHb containing enzymes is imperfect virus inactivation and elimination. An artificial oxygen carrier derived from human red blood cells falls under Specified Biological Product as defined in Japanese pharmaceutical regulations. An artificial oxygen carrier derived from non-human animal red blood cells falls under Biological Product. In both cases, it is required that virus inactivation must be performed in the purification step and that its removal rate (Log Reduction Value) must be sufficiently high compared with a regulatory value. Viral inactivation may be performed by heat treatment in the liquid form (60° C., 10 hours) or S/D treatment (organic solvent/detergent treatment). These treatments inactivate virus by denaturation. In addition, viral elimination may be performed by nanofiltration treatment in which a virus larger than, for example, 15 nm can be eliminated as the virus cannot pass through the filter with the pore size of 15 nm. Hemoglobin is a globular protein and structurally stable, while enzymes are labile. Hemoglobin usually has oxygen bound to its heme. When the oxygen-bound Hb converted to carbon monoxide-bound hemoglobin (HbCO), or to deoxyHb by removing the oxygen, the Hb is rendered thermostable. It is feasible to conduct the heat treatment in the liquid form for viral inactivation. However, almost all enzymes are denatured and inactivated (non-patent documents 4 and 5). The nanofiltration eliminates enzymes larger than the pore size of the nanofiltration filter.

Inventors of the present invention understand that the artificial oxygen carrier (artificial red blood cell) requires complete elimination of the infection sources. Thus, the heat treatment and nanofiltration are conducted in the step of purifying Hb from red blood cells. The viral Log Reduction Value satisfies the regulatory standard, and the product is remarkably safe. On the other hand, the oxygen-carrier function is gradually decreased, because the HbO₂ is converted to metHb with trivalent iron which cannot be reduced to divalent iron, due to the complete loss of the enzyme system after the heat treatment and nanofiltration. Therefore, there is a need to suppress the above-mentioned conversion to metHb in the development of the artificial red blood cell preparation encapsulating purified and concentrated Hb which is substantially free of any enzyme system after the processes of viral inactivation and elimination.

A variety of non-enzymatic metHb reduction systems have been studied. (i) Thiols such as glutathione and cysteine can reduce metHb by direct reaction (non-patent document 6). This method is suitable for complete elimination of remaining oxygen and complete reduction of metHb when stored for a long time under deoxygenated conditions (patent-document 1, non-patent document 7). When the oxygen is present but metHb concentration is not high, conversion to metHb may be adversely promoted due to the direct reaction of the thiols with the dissolved oxygen to produce reactive oxygen species. (ii) Reduction of metHb was conceived by visible light illumination and supplementing the purified and concentrated Hb with flavines as an electron carrier and glucose as an electron donor. The illumination was of limited use and turned out not to be practical (patent document 2). (iii) When amino acid tyrosine is present together with the metHb, it shows pseudoo-catalase activity eliminating hydrogen peroxide. Some degree of suppression of conversion to metHb was observed but failed to be practical (patent document 3). (iv) When NADH and methylene blue were added to the artificial red blood cells (Hb-V), the metHb in the artificial red blood cells were reduced (non-patent document 8). This is interpreted that methylene blue was reacted with NADH to be reduced leukomethylene blue, which was diffused into the artificial red blood cells to reduce metHb. (v) When metHb content is increased after administration of the artificial red blood cells (Hb-V) into blood vessel, a very small dose of methylene blue has a clear efficacy and of practical use in reducing metHb, because the methylene blue in the blood circulation is reacted with NADH or NADPH in the red blood cells and converted to the reduced leukomethylene blue, which is diffused into the artificial red blood cells where metHb is reduced (non-patent document 9). It became problematic that leukomethylene blue may react with oxygen to generate active oxygen and that the color tone of skin turns out to be blue. Therefore, there is a need to develop a technology to delay or suppress conversion to the metHb by an alternative means.

In the blood vessel, nitric oxide (NO) is always released from a blood vessel wall as a vascular endothelium-derived relaxation factor (EDRF). The released NO is extremely reactive with Hb to promote the conversion to metHb. In addition, in an inflammatory response or an ischemic reperfusion injury, the production of NO in the blood vessel is enhanced by induction of the inducible nitric oxide synthase (iNOS), the neutrophils and macrophages are activated, and the action of NADPH-oxidase enhances the production of superoxide, which is dismutated to hydrogen peroxide. It is a problem that they also react with Hb to promote the conversion to metHb. Thus, there is a need to retain a new defense system against excessive endogenous active oxygen and NO to maintain the function of artificial red blood cells in the presence of such oxidative stress in the body. This need has not been recognized in the development of conventional artificial red blood cells or encapsulated Hb.

### PRIOR ART LITERATURE

### Patent Document

Patent document 1: Japanese Patent No. 3466516
Patent document 2: Japanese Patent No. 4181290
Patent document 3: Japanese Patent No. 4763265

### Non-Patent Document

Non-patent document 1: Chang, T M, Biomater Artif Cells Immobilization Biotechnol. 1992; 20(2-4): 159-79.
Non-patent document 2: Natanson C et al., JAMA. 2008; 299(19): 2304-12.
Non-patent document 3: Sakai H et al., Methods Enzymol. 2009; 465: 363-84.
Non-patent document 4: Sakai H et al., Protein Expr Purif. 1993 December; 4(6): 563-9.
Non patent document 5: Sakai H et al., J Biochem. 2002 April; 131(4): 611-7.
Non-patent document 6: Takeoka S et al., Bioconjug Chem. 1997 July-August; 8(4): 539 44.
Non-patent document 7: Sakai H et al., Bioconjug Chem. 2000 May-June; 11(3):425-32.
Non-patent document 8: Takeoka S et al., Artif Cells Blood Subst Immob Biotechnol. 1997; 25: 31-41.
Non-patent document 9: Sakai H et al., Bioconjug Chem. 2014 Jul. 16; 25(7): 1301-10.

### SUMMARY OF THE INVENTION

### Problem to be Solved by the Invention

The problem of the present invention is to provide a non-enzymatic solution which enables to delay or suppress the conversion to metHb, as well as to dissipate positively oxidative stress, not only during storage but also after administration into a patient and during circulating in the blood vessel in the field of artificial red blood cell preparation encapsulating purified and concentrated Hb which is substantially free of any enzyme system.

The inventors contemplated the background and the problem described above and have completed the present invention as a result of intensive studies.

The present invention provides an artificial red blood cell. The artificial red blood cell of the present invention comprises:

an aqueous solution comprising Hb and NADH and/or NADPH; and
a capsule including the aqueous solution,
wherein the aqueous solution and the capsule are substantially free of enzyme activity for reducing metHb.

The artificial red blood cell of the present invention may have a 50% conversion time to metHb of 72 hours or more.

In the artificial red blood cell of the present invention, the concentration of Hb in the aqueous solution included in the capsule may be 10 to 45 g/dL (1.6 to 7.0 mM), and the molar concentration of NADH and/or NADPH in the aqueous solution included in the capsule may be 0.5 to 10 times higher than the molar concentration of Hb in the aqueous solution.

In the artificial red blood cell of the present invention, the encapsulated Hb aqueous solution may comprise pyridoxal 5′-phosphate at a molar concentration 0.5-3 times higher than the molar concentration of the Hb.

In the artificial red blood cell of the present invention, the capsule may be at least one selected from the group consisting of a liposome, a polymersome and a thin film of polymer.

In the artificial red blood cell of the present invention, the liposome may consist of:

1,2-dipalmitoyl-sn-glycero-3-phosphatidylcholine (DPPC),
cholesterol,

### 1,5-O-dihexadecyl-N-succinyl-L-glutamate (DHSG) and

1,2-distearoyl-sn-glycero-3-phosphatidylethanolamine-N-PEG₅₀₀₀(DSPE-PEG₅₀₀₀).

The present invention provides a pharmaceutical preparation for blood surrogate comprising the artificial red blood cell of the present invention

The pharmaceutical preparation for blood surrogate of the present invention comprises of the artificial red blood cell of the present invention wherein the artificial red blood cell is dispersed in an aqueous solution and may further comprise, in the aqueous solution, at least one chemical compound selected from the group consisting of electrolytes, saccharides, amino acids, colloids, NADH and NADPH at a physiologically acceptable concentration.

The present invention provides an agent for eliminating at least one substance selected from the group consisting of NO, H₂O₂, and O₂⁻. The agent comprises a liposome encapsulating an aqueous solution of NADH and/or NADPH. In the agent for eliminating at least one substance selected from the group consisting of NO, H₂O₂, and O₂⁻., the liposome may consist of:

1,2-dipalmitoyl-sn-glycero-3-phosphatidylcholine (DPPC),
cholesterol,

### 1,5-O-dihexadecyl-N-succinyl-L-glutamate (DHSG) and

1,2-distearoyl-sn-glycero-3-phosphatidylethanolamine-N-PEG₅₀₀₀(DSPE-PEG₅₀₀₀).

The present invention provides a pharmaceutical composition for treating sepsis, treating ingestion of a large amount of nitrite salt, and preventing methemoglobinemia during NO inhalation therapy. The pharmaceutical composition of the present invention may be the preparation for blood surrogate of the present invention.

The present invention provides a method for producing an artificial red blood cell. The method for producing an artificial red blood of the present invention comprises the steps of:

(a) substantially eliminating enzyme activity to reduce metHb from a first aqueous solution comprising Hb,
(b) resolving NADH and/or NADPH in the first aqueous solution and preparing a second aqueous solution comprising Hb and NADH and/or NADPH, wherein the Hb is substantially free of enzyme activity for reducing metHb,
(c) encapsulating the second aqueous solution in a capsule and obtain an artificial red blood cell consisting of the second aqueous solution and the capsule.

In the method for producing the artificial red blood cell of the present invention, the step (a) may comprise heating the first aqueous solution at 60 to 65° C. for 1 to 12 hours.

In the method for producing the artificial red blood cell of the present invention, the Hb concentration in the second aqueous solution may be 10 to 45 g/dL (1.6 to 7.0 mM), and the molar concentration of NADH and/or NADPH in the aqueous solution may be 0.5 to 10 times higher than the molar concentration of the Hb.

In the step (b) of the method for producing the artificial red blood cell of the present invention, the second aqueous solution may comprise pyridoxal 5′-phosphate at a molar concentration 0.5-3 times higher than the molar concentration of the Hb.

In the method for producing the artificial red blood cell of the present invention, the capsule may be at least one selected from the group consisting of a liposome, a polymersome and a thin film of polymer.

In the method for producing the artificial red blood cell, the liposome may consist of:

1,2-dipalmitoyl-sn-glycero-3-phosphatidylcholine (DPPC),
cholesterol,

### 1,5-O-dihexadecyl-N-succinyl-L-glutamate (DHSG) and

1,2-distearoyl-sn-glycero-3-phosphatidylethanolamine-N-PEG₅₀₀₀ (DSPE-PEG₅₀₀₀).

In a patient of sepsis, it is known that a large amount of NO is released into the blood circulation, and the percentage of metHb (metHb %, weight percentage of metHb in total Hb) is increased (Ohashi, K et al., Acta. Anaeshesiol. Scand. 42: 713-716 (1998)). In a patient who has ingested a large amount of nitrite, it is known that the percentage of metHb is increased (Cockburn, A et al., Toxicology and Applied Pharmacology, 270: 209-217 (2013); Sharma M K et al., J. Clin. Diagnostic Res. 7: 1552-1554 (2013)). Further, in a neonatal patient who has been subjected to a NO inhalation therapy for the neonatal persistent hypertension, it is known that the percentage of metHb is increased (Salguero, K L et al., Pulmonary Phacol. Therapeutics, 15: 1-5 (2002), Hmon, I et al., Acta Paediatica, 99: 1467-1473 (2010)). As the artificial red blood cell of the present invention suppresses conversion of Hb to metHb even in the presence of nitrite or NO, the pharmaceutical composition of the present invention comprising the artificial red blood cell of the present invention is useful for treating sepsis, treating ingestion of a large amount of nitrite salt, and preventing methemoglobinemia during NO inhalation therapy.

### Effects of the Invention

By encapsulating a predetermined amount of NADH and/or NADPH in an artificial red blood cell, it is feasible to delay or suppress deterioration due to autoxidation and oxidation stress, enabling to retain the oxygen-carrying function for an extended period of time. In addition, it is also feasible to dissipate positively the oxidative stress generated from tissue by ischemia reperfusion injury or inflammatory response.

All the aforementioned patents and documents are incorporated herein by reference in their entirety.

### BRIEF DESCRIPTION OF DRAWINGS

FIG. 1 is a graph showing change over time of the percentage of metHb in artificial red blood cell preparations encapsulating different concentrations of NADH.

FIG. 2 is a graph showing the percentage of metHb in artificial red blood cell preparations encapsulating different concentrations of NADH left to stand in a 37° C. thermostatic bath for 24 hours.

FIG. 3A is a graph showing change over time in absorbance at 630 nm after adding NOC7 to artificial red blood cells encapsulating NADH.

FIG. 3B is a graph showing change over time of the percentage of metHb in artificial red blood cell preparations after adding NOC7 to artificial red blood cells encapsulating NADH

FIG. 4A is a graph showing change over time in absorbance at 630 nm after adding H₂O₂ to artificial red blood cells encapsulating NADH.

FIG. 4B is a graph showing change over time of the percentage of metHb in artificial red blood cell preparations after adding H₂O₂ to artificial red blood cells encapsulating NADH

FIG. 5A is a graph showing change over time in absorbance at 630 nm after adding NaNO₂ to artificial red blood cells encapsulating NADH.

FIG. 5B is a graph showing change over time of the percentage of metHb in artificial red blood cell preparations after adding NaNO₂ to artificial red blood cells encapsulating NADH.

FIG. 6A is a graph showing change over time in absorbance at 630 nm after adding NOC7 to an Hb solution supplemented with NADH.

FIG. 6B is a graph showing change over time of the percentage of metHb after adding NOC7 to a Hb solution supplemented with NADH.

FIG. 7A is a graph showing change over time in absorbance at 630 nm after adding H₂O₂ to a Hb solution supplemented with NADH.

FIG. 7B is a graph showing change over time of the percentage of metHb after adding H₂O₂ to a Hb solution supplemented with NADH.

FIG. 8A is a graph showing change over time in absorbance at 630 nm after adding NaNO₂ to an Hb solution supplemented with NADH.

FIG. 8B is a graph showing change over time of the percentage of metHb after adding NaNO₂ to an Hb solution supplemented with NADH.

FIG. 9 is a graph showing change over time in NADH concentration (absorbance at 340 nm) after mixing NADH and NOC 7.

FIG. 10 is a graph showing change over time in NADH concentration (absorbance at 340 nm) after mixing NADH and H₂O₂.

FIG. 11 is a graph showing change over time in NADH concentration (absorbance at 340 nm) after mixing NADH and NaNO₂.

FIG. 12 is a graph showing change over time of the percentage of metHb after administration to rats of artificial red blood cells encapsulating NADH.

FIG. 13 is a graph showing change over time of the percentage of metHb after administration to rats with hemorrhagic shock of artificial red blood cells encapsulating NADH.

### DETAILED DESCRIPTION OF THE INVENTION

Examples of the present invention described below are intended to be illustrative only, and not intended to limit the technical scope of the present invention. The technical scope of the present invention is limited only by the claims. Without departing from the spirit and scope of the present invention, various modifications of the present invention may be made for example, the addition, deletion and replacement of features of the present invention

In the present specification, an “artificial red blood cell” means a capsule encapsulating Hb (Hb), which is used as an alternative to a red blood cell derived from circulating blood of human and other animals. The main function of blood is oxygen transportation, and Hb in a red blood cell reversibly binds to oxygen. A solution with chemically modified Hb has been developed as an alternative to blood for transfusion. However, the Hb solution directly put in the blood circulation is found to be toxic. The explanation is that the Hb has a strong affinity or reactivity to nitric oxide (NO), a vascular endothelial relaxation factor, and inactivate NO, leading to vasoconstriction and peripheral circulation failure, that Hb causes various side effects because the Hb is easily leaked out of the blood vessel due to the small particle size (Natanson C et al., JAMA. 2008; 299 (19): 2304-12). This is tantamount to the toxicity of free Hb released by hemolysis. Thus, encapsulated Hb has been developed as artificial red blood cells. In addition, artificial red cells encapsulating HbCO may be administered, because it has been demonstrated from an animal experimental model that the artificial red blood cells are effective as CO carrier or the like. CO acts on a heme protein involved in active oxygen production and suppresses production of active oxygen. In this case, CO is gradually dissociated in the blood vessel from the Hb, which is converted to HbO₂, an oxygen carrier. Therefore, although deterioration does occur due to autoxidation and oxidation stress, the oxygen carrying function can be retained for an extended period of time by allowing NADH and/or NADPH to coexist in the artificial red blood cells.

The Hb encapsulated in the artificial red blood cells of the present invention may be purified and concentrated from human red blood cells and red blood cells of domestic animals. Alternatively, purification and concentration of the Hb may be carried out using product of a microorganism using the recombinant DNA technology. Any purification and concentration processes known to those skilled in the art may be carried out including, but not limited to, the following processes for example. Human or livestock blood is centrifuged to remove supernatant of the plasma and buffy coat. The precipitate is gently mixed with isotonic solution (saline or phosphate buffered saline), and centrifuged to remove the supernatant. This washing process is repeated three times to obtain the washed red blood cells. Addition of hypotonic solution (such as pure water) induces hemolysis of the red blood cells and Hb is released. Membrane components are removed either by precipitation with ultracentrifuge or by filtrating only Hb with an ultrafiltration membrane (molecular weight cut-off of about 1000 kDa). Obtained by the process of viral inactivation and elimination as explained below, HbCO solution is subject to dialysis and pH adjustment and then concentrated with an ultrafiltration membrane. In order to carry out efficiently, the molecular weight cut-off of the latter ultrafiltration membrane may be about 8,000 to 30,000. The obtained HbCO solution is thick with Hb concentration of 30 to 45 g/dL. This solution is subjected to anion exchange resin treatment, and is filtrated with a sterilization filter having a pore sizer of 0.22 μm.

In the artificial red blood cells of the present invention, the material of the capsule including Hb is a polymer film prepared with material comprising, but not limited to, polymer such as polystyrene, gum arabic, nylon, and silicone; a material derived from a living organism such as gelatin; a copolymer of poly (epsilon-caprolactam) or polyethylene glycol with a biodegradable polymer such as polylactic acid and polyglycolic acid; polysaccharide, a copolymer of an amino acid polymer. Furthermore, the material of the capsule may comprise, but not limited to, hydrogel, silica gel, Hb/O/w emulsion, heparin-poly alkyl cyanoacrylate, polyion complex, polymersome, niosome and liposome.

When the capsule including Hb in the artificial red blood cells of the present invention is liposome, it is excellent in biocompatibility and easy to prepare, because Hb is included in the lipid bilayer like the biological membrane (Djordjevici L et al., Fed Proc 1977, abstract No. 1561 (physiology), page 567). It has been reported that various lipid compositions are used for liposome (Sakai H et al., Methods Enzymol. 2009; 465: 363-84). In an example of an artificial red blood cell encapsulating Hb in a liposome, the liposome consists of four components, 1,2-dipalmitoyl-sn-glycero-3-phosphatidylcholine (DPPC), cholesterol, 1,5-O-dihexadecyl-N-succinyl-L-glutamate (DHSG), and 1,2-distearoyl-sn-glycero-3-phosphatidylethanolamine-N-PEG₅₀₀₀(DSPE-PEG₅₀₀₀). In the liposome consisting of the above-mentioned components, concentration of Hb in the dispersion solution of Hb vesicles (Hb-V) is 10 to 45 g/dL, which corresponds to molar concentration of 1.6 to 7.0 mM. Administration tests with animals have confirmed a high safety level of the artificial red blood cell (Hb-V). In addition, animal model experiments have demonstrated that the artificial red blood cell has efficacy in resuscitation from hemorrhagic shock, high level of hemodilution, administration for anemia therapy, reduction of infarct region of cerebral infarction, oxygenation in the ischemic region, supplement for extracorporeal circulation circuit, an organ perfusion solution, a CO carrier, and others.

It has been known that the means for preparing liposome comprises ultrasonic treatment (probe method, bath method), organic solvent injection, detergent removal, freeze-thawing, reverse phase evaporation, extrusion, rehydration of dried liposome powder, high-pressure emulsion dispersion, kneading by planetary movement, and others. To encapsulate Hb without denaturing Hb, it is preferable to carry out rehydration of dried liposome powder, extrusion, kneading and others.

In the present invention, NADH and/or NADPH may be added to the Hb solution encapsulated in the artificial red blood cell of the present invention at a molar ratio of 0.5 to 10 times more than the Hb. Alternatively, NADH and/or NADPH may be added at a molar ratio of 1 to 3 times more than the Hb. The relationship of the molar concentrations of NADH, NADPH, and Hb encapsulated in the artificial red blood cell of the present invention may be defined as follows:

0.5≤([NADH]+[NADPH])/[Hb]≤10

or

1≤([NADH]+[NADPH])/[Hb]≤3

wherein [NADH], [NADPH] and [Hb] represent the molar concentration of NADH, NADPH and Hb, respectively. In the present invention, it has been well-known to those skilled in the art that “NADH and/or NADPH” can be replaced with “an enzyme system capable of converting NAD⁺ and/or NADP⁺ to NADH and/or NADPH and NAD⁺ and/or NADP⁺”.

In addition to NADH and/or NADPH, an additive may be added to the Hb solution included in the capsule. The additive includes, but not limited to, pyridoxal 5′-phosphate (PLP). PLP may be added as allosteric factor for adjusting the oxygen affinity of Hb at a molar ratio of 0-3 times more than Hb. Alternatively, the relationship of the molar concentrations of PLP, and Hb encapsulated in the artificial red blood cell of the present invention may be defined as follows:

0<[PLP]/[Hb]≤3

After the encapsulation step, Hb which has not been encapsulated in the capsule may be filtered and removed with treating ultrafiltration membrane (for example, molecular weight cut-off of about 1000 kDa). Alternatively, the artificial red blood cells are precipitated by a centrifuge to remove supernatant Hb solution, and saline or the like may be added to the precipitate to disperse the artificial red blood cells. The redispersion solution of the artificial red blood cells may comprise an additive including, but not limited to, an electrolyte, a carbohydrate, a colloid, an amino acid, NADH and NADPH. The colloid as used herein refers to any substance which can provide colloid osmotic pressure to the dispersion solution of the artificial red blood cells including, but not limited to, albumin (5 g/dL or less), hydroxyethyl starch (10 g/dL or less), dextran (10 g/dL or less), modified gelatin (5 g/dL or less). The crystalloid osmotic pressure is preferably adjusted to 300 mOsm. NaCl concentration of normal saline is 0.9 wt %, however, it is possible to augment the resuscitation effect by elevating the NaCl concentration to around 7 wt % to be hyperosmolar. When the dispersion solution of the artificial red blood cells comprises NADH and/or NADPH, it can be expected to exert further protecting effect of the Hb.

When the HbCO solution is encapsulated, the HbCO is required to be converted to HbO₂ by a photoreaction under oxygen gas flow. The reaction is accelerated by forming a liquid film, or irradiating visible light while permeating through the dialysis membrane (Japanese Patent Publication No. 3682072)

The “enzyme for reducing metHb” herein includes, but not limited to, a nicotinamide adenine dinucleotide (NADH)-based metHb reductase, which uses NADH as substrate, and NADPH-based metHb reductase. Although NADH-metHb reductase was reported as a NADH-based metHb reductase, it was subsequently demonstrated that its mechanism involves reduction of metHb by an action of NADH-cytochrome b₅ reductase with cytochrome b₅ as an electron mediator. The oxidized form, NAD⁺, is restored to NADH by the Embden-Meyerhof pathway. There are two type of NADH-cytochrome b₅ reductases, one, present on the red blood cell membrane, and another, resolved in red blood cells. The NADPH-based metHb reductase reduces metHb by the action of NADPH metHb reductase using nicotinamide adenine dinucleotide phosphate (NADPH) as substrate. The oxidized form, NADP⁺ is restored to NADPH by the pentose phosphate pathway. The “enzyme system capable of converting NAD⁺ to NADH” and “enzyme system capable of converting NADP⁺ to NADPH” of the present invention refer to glyceraldehyde 3-phosphate dehydrogenase (GAPDH) of the Embden-Meyerhof pathway and glucose-6-phosphate dehydrogenase (G6PDH) and 6-phosphogluconate dehydrogenase (6PGDH) of the pentose phosphate pathway, respectively. Accordingly, the “enzyme for reducing metHb” and “enzyme system capable of converting NAD⁺ and/or NADP⁺ to NADH and/or NADPH” are distinct from each other.

For the enzyme for reducing metHb herein, “substantially free of enzyme activity” means that the enzyme activity is 10%, 5%, 3%, 1%, 0.5%, 0.2%, 0.1%, 0.05% or 0.03% or less, or below detectable limits, of enzyme activity of the enzyme for reducing metHb existing in the Hb solution purified and concentrated from blood derived from humans or domestic animals. The enzyme activity of the enzyme for reducing metHb herein disclosed herein is determined, for example, by the following procedure. Determination of NADH-cytochrome b₅ reductase may be carried out according to Beutler, E. (RED CELL METABOLISM: A MANUAL OF BIOCHEMICAL METHOD, Grune &Stratton, Inc., pp. 81-82 (1987)). The principle of determination of this method is to measure NADH metHb reductase activity as NADH-ferricyanide reductase activity.

In the reaction of the following formula:

K₃Fe(CN)₆+NADH+H⁺→K₃HFe(CN)₆+NAD⁺

the enzyme activity is measured by tracing the degree of acceleration by adding sample comprising oxygen. The measurement is carried out by measuring the change (decrease) in absorbance at 340 nm which is the characteristic absorption of the consumed NADH.

Determination of NADPH-flavin reductase activity may be carried out according to Beutler, E. (RED CELL METABOLISM: A MANUAL OF BIOCHEMICAL METHOD, Grune &Stratton, Inc., pp. 79-80 (1987)). The principle of determination of this method is to measure NADH metHb reductase activity as NADH diaphorase activity. The reaction of reducing methylene bleu (MeBl) to leucomethylene blue (LeukMeBl) as follows:

NADPH+H⁺+MeBl→NADP⁺+LeukMeBl

the reaction is known to be slow. The enzyme activity is measured by tracing the degree of acceleration by adding sample comprising oxygen. The measurement is carried out based on the above-mentioned principle by measuring the change (decrease) in absorbance at 340 nm which is the characteristic absorption of the consumed NADPH.

The condition where the enzyme for reducing metHb “substantially free of enzyme activity” as disclosed herein may be achieved by purification and concentration of Hb derived from human or livestock blood, followed by heat treatment for viral inactivation and/or nanofiltration treatment for viral elimination. The heat treatment for viral inactivation may be carried out, for example, by heating the purified and concentrated Hb solution for 10 to 12 hours at 60° C. The nanofiltration treatment for viral elimination may be carried out, for example, by nanofiltration of the Hb solution with a nanofiltration membrane with a pore size of 15 to 50 nm, after the above-mentioned purification and concentration treatment and further by the heat treatment for viral inactivation.

By the heat treatment and nanofiltration, the total of the ratios of viral inactivation and elimination (log reduction value) exceeds 9, clearing the regulations required as virus clearance validation. These treatments completely eliminate all the enzymes involved in glycolysis and reduction of metHb, decarboxylases, and enzymes removing reactive oxygen. In particular, the NADH-cytochrome b₅ reductase using NADH as substrate and its electron mediator, cytochrome b₅, are denatured and inactivated, because they have been well known as thermolabile (Arinc E. et al., Comp Biochem Physiol B. January-February; 101 (1-2): 235-42. (1992)). The protein purity of the resulting Hb solution becomes extremely high (99.8% or more). It is confirmed that the Hb solution does not show enzymatic activities that correspond to NADH-cytochrome b₅ reductase and NADPH-flavin reductase.

In the present specification, “50% metHb conversion time” used herein refers to the time it takes for 50% of the measured Hb, which is encapsulated in the artificial red blood cells, to be converted to metHb. The 50% metHb conversion time is determined by the following procedures.

Aliquots of the Hb solution are collected as specimens at the start of the incubation and thereafter, for example, 0, 2, 6, 8, 24 and 26 hours after incubation, and they are subjected to the determination of the 50% metHb conversion time. An ultraviolet visible spectrophotometer (V-660; Jasco Corp. Tokyo, Japan) with built-in integrating sphere is used for the determination. The specimens are diluted with saline, sealed in a cuvette, and deoxygenized by nitrogen bubbles, to make a two-component system of deoxyHb and metHb. The metHb conversion percentage is calculated from the ratio of absorbances at maximum absorption wavelength of 430 nm and 405 nm. The 50% metHb conversion time is determined from a graph of time-course measurements until the metHb conversion percentage exceeds 50%. In case the metHb conversion percentage does not reach to 50%, the time to convert 50% of Hb to metHb is determined by extrapolating from the graph of time-course measurements.

The agent for eliminating NO and H₂O₂ of the present invention includes a liposome encapsulating aqueous solution of NADH and/or NADPH. As the NADH and NADPH are encapsulated in a liposome, they cannot contact an enzyme, and thus, are not consumed as a coenzyme of an enzymatic reaction. However, it has been known that the lipid membrane of the liposome does not have barrier properties with regard to small molecules such as NO and H₂O₂(Sakai H et al., J Biol Chem. 2008 Jan. 18; 283(3): 1508-17; Takeoka S et al., Bioconjug. Chem. 2002 November-December; 13(6): 1302-8). By retaining aqueous solution of NADH and/or NADPH in a liposome, therefore, it can be expected that the residence time in blood circulation for NADH and/or NADPH is greatly prolonged, for example, when administered into a blood vessel, exerting an effect to eliminate NO and H₂O₂ generated excessively in the blood vessels.

### EXAMPLE

### Example 1

Human red blood cells in eight bags of expired human red blood cell (each containing 400 ml of donated blood for transfusion) were poured into plastic centrifuge bottles (500 mL) and centrifuged (3,000 rpm, 1 hour). Remaining plasma components and buffy coat (white blood cells) in the supernatant were withdrawn by suction with an aspirator. Then, the bottles were filled with saline for injection and lightly agitated, centrifuged again under the same condition as disclosed in the above and the supernatant was withdrawn by suction. This process is repeated for two more times to obtain washed human red blood cells. The washed red blood cells were subjected to tangential flow ultrafiltration (molecular weight cut off: 1,000 kDa) while adding distilled water for injection. The Hb released by hemolysis was filtered and collected, with the red blood cell membrane components (stroma) separated and removed. The recovered filtrate is referred as stroma free Hb, or SFHb, which still includes soluble enzyme proteins resolved in the red blood cells, although the red blood cell membrane components were eliminated. The SFHb, after concentrated with ultrafiltration membrane (molecular weight cut off: 8 kDa) up to around 10 to 20 g/dL, was transferred to a sealed thermoresistant vessel, was repeatedly subjected to filling with carbon monoxide gas and agitating, to convert oxyHb (HbO₂) to carbonyl Hb (HbCO). The Hb solution was heated to 60° C. for 12 hours while agitating gently with a propeller stirrer. This heat treatment is for viral inactivation, but also serves to denature and insolubilize contaminating proteins. After confirming that the temperature of the solution returned back to room temperature, the Hb solution was centrifuged to remove the denatured and insoluble proteins as precipitate and treated with ultrafiltration membrane (molecular weight cut off: 1,000 kDa). The filtrate was immediately subjected to filtration for viral elimination (nanofiltration treatment), and then, to dialysis with an ultrafiltration membrane (molecular weight cut off: 8 kDa) until the salt concentration (NaCl concentration was calculated based on Na⁺ concentration) is below 0.01% or lower. The Hb solution was further filter-concentrated up to 40 to 42 g/dL, treated with anion exchange resin, and filtrated with a negatively-charged sterile filter with pore size of 0.22 μm to obtain purified and concentrated HbCO solution. The purified and concentrated HbCO solution were subjected to examination of enzyme activities and screening of effect to suppress HbO₂ autoxidation below, as well as to encapsulating step as described in Example 2.

The activity of NADH-cytochrome b₅ reductase in the above-mentioned purified and concentrated HbCO solution was determined as NADH-ferricyanide reductase activity according to Beutler, E. above. Diluted Hb samples comprising the enzyme to be measured is mixed with a buffer comprising NADH. The mixed solutions were left standing for 10 minutes at 30° C., and then potassium ferricyanide (K₃Fe (CN)₆), the enzyme substrate, was added to start the enzyme reaction. The decrease of difference of absorbance at 340 nm, a characteristic absorption of NADH, per minute was measured to determine activity unit E (IU/gHb). A control crude SFHb solution had 20 IU/gHb activity, while the purified and concentrated Hb did not detect any activity. It was reasoned that the heat-labile NADH-cytochrome b₅ reductase was eliminated as denatured and insoluble, or denatured and inactivated, due to the introduction of heat treatment during the purifying step. In addition, activities of NADPH metHb reductase and NADPH diaphorase, which correspond to NADPH-flavin reductase are also determined by the above-mentioned methods according to Beutler, E. Diluted blood samples comprising the enzyme to be measured is mixed with a buffer comprising NADPH. The mixed solutions were left standing for 10 minutes at 37° C., and then methylene bleu (MeBl), the enzyme substrate, was added to start the enzyme reaction. The decrease of difference of absorbance at 340 nm, a characteristic absorption of NADPH, per minute was measured to determine activity unit E (IU/gHb). A control crude SFHb solution had 2.91 IU/gHb activity, while the purified and concentrated Hb did not detect any activity (below 0.01 IU/gHb). Catalase activity of the purified HbCO solution was determined from the change of the absorbance at 230 nm, the absorption wavelength of H₂O₂, according to Beutler, E. (RED CELL METABOLISM: A MANUAL OF BIOCHEMICAL METHOD, Grune &Stratton, Inc. pp. 105-106 (1987)). A control crude SFHb solution had 17.5×10⁴ IU/gHb activity, while the purified and concentrated Hb did not detect any activity (0.03×10⁴ IU/gHb or less). Pyrogallol was used for measurement of SOD activity (Marklund, S. and Marklund, G., Eur. J. Biochem. 47, 469-474 (1974); Roth, E. F. Jr. and Gilbert, H. S., Anal. Biochem. 137, 50-53(1984)). Pyrogallol is oxidized with oxygen to generate superoxide, and further reacted to generate a yellow dye with absorbance at 420 nm. SOD inhibits this reaction. SOD enzyme activity was determined based on this inhibitory activity. Analyzed specimens were solutions of purified HbO₂, HbCO, metHb and solutions thereof further comprising NADH or L-tyrosine In addition, enzyme activity of crude SFHb solution was also determined as a control. High catalase and SOD activities were detected in the control crude SFHb solution, no enzyme activity was found in HbO₂, metHb and HbCO prepared from the purified Hb solution used for the present experiments. When NADH and HbO₂ were coexisted, however, high CAT and SOD pseudo activities (Table 1). When tyrosine and HbO₂ were coexisted, the pseudo activities were detected at extremely low level compared with NADH. The pseudo activity was detected when NADH alone was present, however, the activity was about a tenth ( 1/10) compared with when HbO₂ was coexisted. It was reasoned that Hb must have divalent iron and form dioxygen complex, as only trace pseudo activity was detected when NADH was coexisted with metHb or HbCO. There were results showing that this pseudo activity increased as more NADH was added. Further, the absence of contaminating protein was confirmed, as analyses with HPLC, isoelectronic focusing and SDS-PAGE did not detect any protein other than Hb.

TABLE 1

| | | | | antioxidant | | |
|---|---|---|---|---|---|---|
| Entry | | Hb | antioxidant | conc. | pseudo CAT | pseudo SOD |
| No. | Hb form | (mM) | contained | (mM) | (IU/g Hb) | (U/g Hb) |
| 1 | HbO₂ | 1.5 | NADH | 1.5 | 34.3 × 10⁴ | 15.6 × 10² |
| 2 | HbO₂ | 1.5 | NADH | 3.0 | 35.2 × 10⁴ | 16.0 × 10² |
| 3 | HbO₂ | 1.5 | NADH | 15.0 | 46.8 × 10⁴ | 20.4 × 10² |
| 4 | metHb | 1.5 | NADH | 1.5 | 4.5 × 10⁴ | 1.2 × 10² |
| 5 | HbCO | 1.5 | NADH | 1.5 | 0.5 × 10⁴ | 1.0 × 10² |
| 6 | HbO₂ | 1.5 | — | 1.5 | N.D. | N.D. |
| 7 | metHb | 1.5 | — | 1.5 | N.D. | N.D. |
| 8 | HbO₂ | 1.5 | — | 15.0 | 0.3 × 10⁴ | 0.2 × 10² |
| 9 | metHb | 1.5 | — | 15.0 | 0.2 × 10⁴ | 0.1 × 10² |
| 10 | — | | NADH | 1.5 | 3.6 × 10⁴ *) | 1.1 × 10² |
| 11 | — | | NADH | 3.0 | 4.1 × 10⁴ *) | 1.3 × 10² |
| 12 | — | | NADH | 15.0 | 19.2 × 10⁴ *) | 0.6 × 10² |
| 13 | HbO₂ | 1.5 | — | — | N.D. | N.D. |
| 14 | metHb | 1.5 | — | — | N.D. | N.D. |
| 15 | HbCO | 1.5 | — | — | N.D. | N.D. |
| 16 | SFHL(HbO₂)**⁾ | 1.5 | — | — | 21.1 ± 1.4 × 10⁴ | 21.6 ± 1.7 × 10² |

*⁾ each NADH conc. was calculated relative to Hb conc. of 1.5 mM (10 g/dL)
**⁾stroma free homolysate SFHL (n = 3)
N.D.: not detected

Potential antioxidant used to study suppressing effect of various compounds against autoxidation of HbO₂, included one protein: human albumin preparation (“Benesis”, 25% albumin from donated blood for iv injection, 5 g/20 mL, Japanese Blood Products Organization (ALB)); nine amino acids: L-tyrosine, L-arginine, L-glutamine, L-tryptophan, L-lysine, L-histidine, L-asparagine, L-cysteine and L-methionine, two antioxidants: quercetin and astaxanthin; water-soluble polymers: PEG₂₀₀₀, PEG₄₀₀, PEG₂₀₀, and hydroxyethyl starch; and antioxidants considered to be less toxic: sodium L-Ascorbate, D-glucose, sodium gluconate, glycerol, nicotinamide, ATP, NADPH, and NADH. NADH was purchased from Oriental Yeast Co. Ltd., and all the rest were purchased from Sigma-Aldrich (St. Louis, Mo., USA). HbCO solution was transferred to an eggplant flask and irradiated with visible light (halogen lamp; LPL Videeoligtvl-302, LPLCO. Ltd., Tokyo, Japan) under oxygen gas flow to convert to HbO₂. After diluting with saline to 10 g/dL, the HbO₂ solution was supplemented with one of the above 25 kinds of antioxidants at the concentration of 1 g/dL, and the conversion ratios to metHb were determined after incubation at 37° C. for 24 hours. The conversion ratios to metHb were determined from the visible absorption spectrum. The measurement of spectrum between 300 nm to 500 nm was conducted using a spectrophotometer (V-660, Japan Spectroscopic Company (JASCO)) mounted with a device to control the light scattering to the minimum (an integrating sphere). The atmosphere inside of a Thunberg cuvette was replaced with nitrogen gas and the conversion ratios to metHb were determined from the ratios of absorbance at 405 nm (λₘₐₓ of metHb) and 430 nm (λₘₐₓ of deoxyHb).

TABLE 2

| antioxidant | metHb conversion (%) |
|---|---|
| no supplementation | 53 |
| L-tyrosine | 38 |
| L-arginine | 38 |
| L-glutamine | 43 |
| L-tryptophan | 38 |
| L-lysine | 34 |
| L-histidine | 32 |
| L-asparagine | 40 |
| L-cysteine | 34 |
| L-methionine | 41 |
| human albumin | 32 |
| PEG₂₀₀₀ | 42 |
| PEG₄₀₀ | 43 |
| PEG₂₀₀ | 39 |
| hydroxyethyl starch | 37 |
| quercetin | 42 |
| astaxanthin | 39 |
| sodium gluconate | 49 |
| sodium L-Ascorbate | 37 |
| D-glucose | 36 |
| glycerol | 38 |
| nicotinamide | 34 |
| ATP | 44 |
| NADPH | 37 |
| NADH | 17 |

The results on the suppressing effects of the above 25 kinds of compounds against HbO₂ autoxidation were summarized in Table 2 above. The metHb conversion percentage of HbO₂ which does not contain any enzyme increased as high as 53% by the autoxidation after incubation at 37° C. for 24 hours. Addition of one of the nine amino acids suppressed the conversion ratio down to 32 to 43%. The conversion ratio of the human albumin preparation was 32% and the conversion ratios of PEG₂₀₀₀, PEG₄₀₀₀, PEG₂₀₀, and hydroxyethyl starch were 37 to 43%. Of the other compounds, nine of them, quercetin, astaxanthin, sodium gluconate, sodium L-Ascorbate, D-glucose, glycerol, nicotinamide, ATP and NADPH suppressed the conversion ratios to 34 to 49%. In contrast, only NADH remarkably decreased the conversion ratio to 17%, demonstrating excellent function of suppressing the metHb conversion percentage.

### Example 2

To 10 mL of the above-mentioned purified and concentrated HbCO solution (41 g/dL or 6.2 mM), an amount of pyridoxal 5′-phosphate (Aldrich, PLP) equimolar to Hb was added. Then, 0, 3.1, 6.2, 12.4 or 24.8 mM of NADH (Boehringer, sodium salt) was added, corresponding to the molar ratio to NADH of 0, 0.5, 1.0, 2.0 or 4.0, respectively. In a cylindrical plastic vessel (volume: 50 mL), powder of mixed lipids (DPPC/cholesterol/DHSG/DSPE-PEG₅₀₀₀) were added to the HbCO solution. The lipids were dispersed by gently rolling of the vessel on a roller shaker, liposomes were formed, and the dispersion solution of the artificial red blood cells were simply prepared. The artificial red blood cells were precipitated by ultracentrifuge separation (50,000×g, 1 hour) to discard the supernatant containing Hb solution which had not been encapsulated. The artificial red blood cells were dispersed by adding saline to the precipitate, and the Hb concentration of the dispersion solution was adjusted to 9.9 g/dL. HbCO was converted to HbO₂, by the light irradiation of the dispersion solution on the ice under oxygen gas flow according to the conventional art.

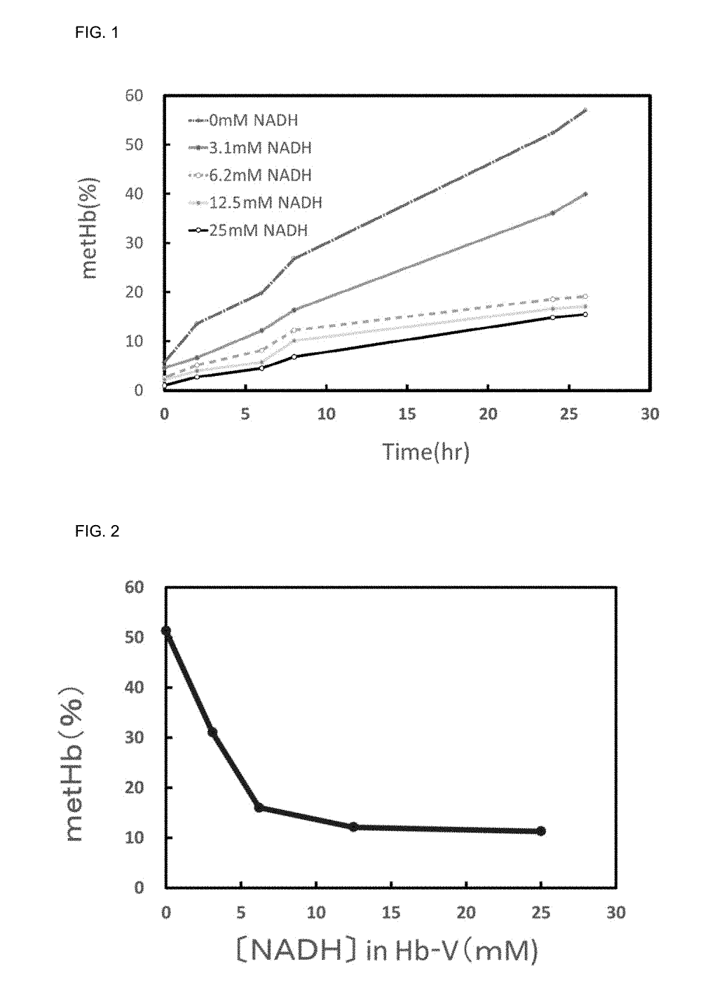

For each of the artificial red blood cells encapsulating different amount of NADH, the metHb conversion percentage was determined by collecting aliquots after incubation for 0, 2, 6, 8, 24 and 26 hours. An ultraviolet visible spectrophotometer (V-660; Jasco Corp. Tokyo, Japan) with built-in integrating sphere was used as the measurement device. The specimens were diluted with saline, sealed in a cuvette, and deoxygenized by nitrogen bubbling, to make a two-component system of deoxyHb and metHb. The metHb conversion percentage was calculated from the ratio of absorbance at maximum absorption wavelength of 430 nm and 405 nm, of deoxyHb and metHb, respectively. The results showed that the higher the NADH concentration encapsulated in the artificial red blood cells, the slower the progression of the conversion to metHb (FIG. 1). The metHb conversion percentage is put on the vertical axis, and incubation time is put on the horizontal axis of the graph of FIG. 1. The five lines represent the changes over time of the metHb conversion percentage of the artificial red blood cells encapsulating 0, 3.1, 6.2, 12.5 and 24 mM of NADH, respectively. From the changes over time of the metHb conversion percentage, the 50% metHb conversion time of the artificial red blood cells encapsulating 0, 3.1, 6.2, 12.5 and 24 mM of NADH are determined as 22, 35, 72, 74 and 76 hours, respectively. When the graph of FIG. 1 is converted to a graph in which the concentration of added NADH is put on the horizontal axis and the metHb conversion percentage after 24 hours is put on the vertical axis, it is clear that the conversion to metHb is suppressed when the molar concentration of the added NADH encapsulated in the artificial red blood cells is approximately identical to, or higher than, the molar concentration of the encapsulated Hb (FIG. 2). In FIG. 2, the percentage (%) of conversion to metHb after 24 hours is put on the vertical axis, and the concentration (mM) of NADH added to the artificial red blood cells ([NADH] in Hb-V) is put on the horizontal axis. Accordingly it is considered that the optimum condition is adding NADH at concentration approximately identical to or higher than the molar concentration of the Hb.

### Comparative Example 2

Artificial red blood cells were prepared which were based on those of Example 2, but which did not comprise PLP, and whose molar ratio of NADH to Hb was 1.0. When the metHb conversion ratios were determined for aliquots collected from the artificial red blood cells left standing in the 37° C. thermostatic bath, the conversion to metHb was found to be accelerated compared with those which comprised PLP.

### Example 3

Following experiment was carried out with those artificial red blood cells prepared in Example 2 whose molar ration of NADH to Hb was 1.0.

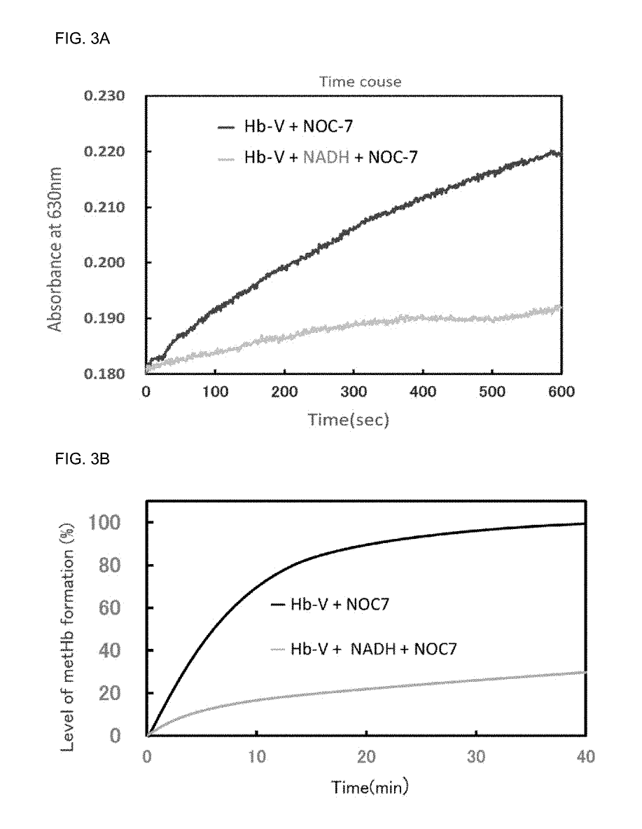

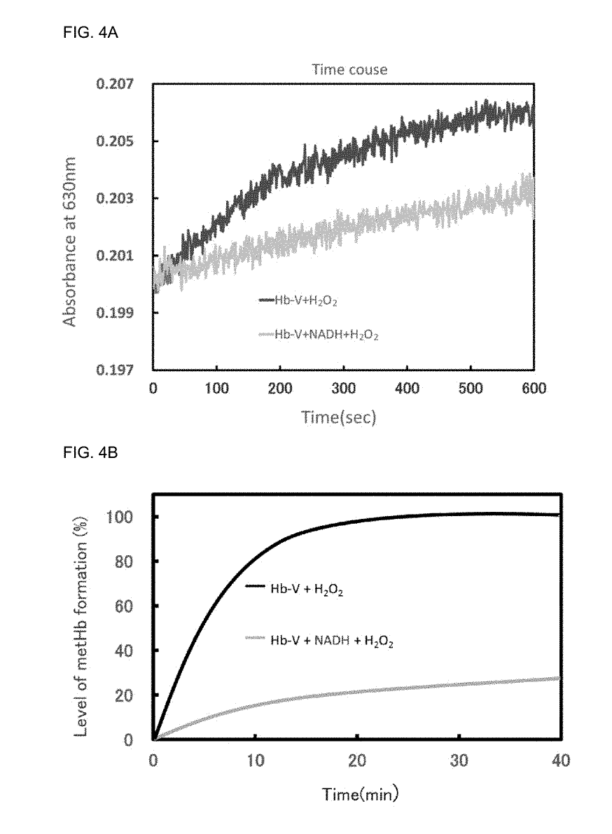

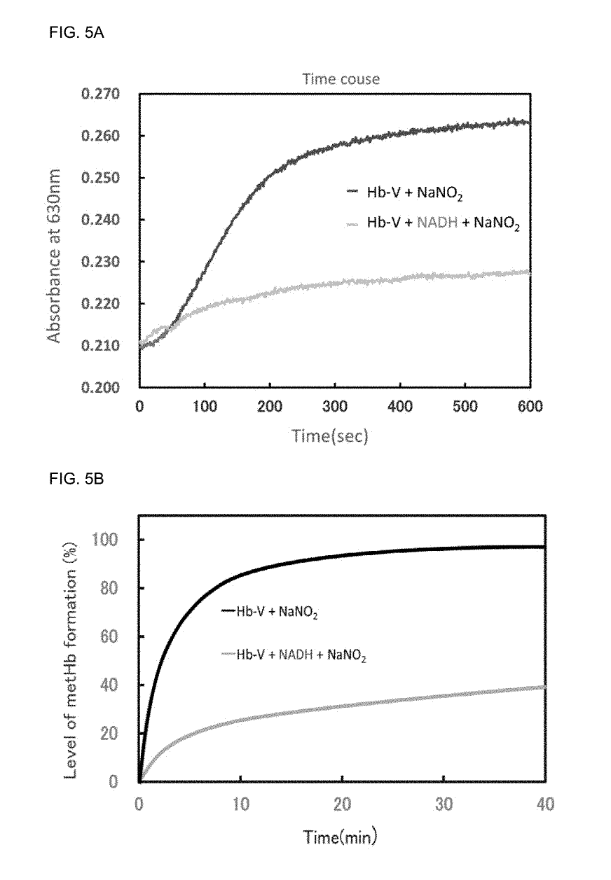

(1) Nitric oxide (NO), a vascular endothelium-derived relaxation factor, is known to react rapidly with Hb to promote conversion to metHb. Thus, the conversion to metHb was studied in the presence of NOC 7 (1-hydroxy-2-oxo-3-(N-methyl-3-aminopropyl)-3-methyl-1-triazene, DOJINDO LABORATORIES) which generates NO. To a cuvette with an optical path length of 1 cm, 3 mL of phosphate buffered saline (PBS, GIBCO, pH 7.4), 30 μL of dispersion solution of the artificial red blood cells encapsulating NADH (Hb concentration: 9.9 g/dL or 1.5 mM) and 30 μL of NOC (1.5 mM in PBS) were mixed and the change of absorbance at 630 nm, the absorption wavelength of metHb, at 25° C. was immediately monitored for 10 minutes and for 40 minutes (FIGS. 3A and 3B). In the graphs of FIGS. 3A and 3B, the absorbance at 630 nm was put on the vertical axis and incubation time (second) was put on the horizontal axis. The two lines in FIGS. 3A and 3B represent a change over time of metHb when the dispersion solution encapsulating NADH was supplemented with NOC7 (Hb-V+NADH+NOC7) and a change over time of metHb when the dispersion solution encapsulating no NADH was supplemented with NOC7 (Hb-V+NOC7). Increase of metHb-specific absorbance at 630 nm was remarkably suppressed when NADH was encapsulated compared with the increase when NADH was not encapsulated. It was interpreted that metHb conversion was suppressed by encapsulating NADH, as NADH partially removed the effect of NO (NOC7) which promotes the metHb conversion.
(2) Next, the effect of H₂O₂, a species of reactive oxygen, known to promote metHb conversion was studied. To a cuvette with an optical path length of 1 cm, 3 mL of phosphate buffered saline (PBS, GIBCO, pH 7.4), 30 μL of dispersion solution of the artificial red blood cells encapsulating NADH (Hb concentration: 9.9 g/dL or 1.5 mM) and 30 μL of H₂O₂ (1.5 mM) were mixed sand the change of absorbance at 630 nm, the absorption wavelength of metHb, at 25° C. was immediately monitored for 10 minutes and for 40 minutes (FIGS. 4A and 4B). In the graphs of FIGS. 4A and 4B, the absorbance at 630 nm was put on the vertical axis and incubation time (second) was put on the horizontal axis. The two lines in FIGS. 4A and 4B represent a change over time of metHb when the dispersion solution with encapsulated NADH was supplemented with H₂O₂ (Hb-V+NADH+H₂O₂) and a change over time of metHb when the dispersion solution without encapsulated NADH was supplemented with H₂O₂(Hb-V+H₂O₂). Increase of metHb-specific absorbance at 630 nm was remarkably suppressed when NADH was encapsulated compared with the increase when NADH was not encapsulated. It was interpreted that metHb conversion was suppressed by encapsulating NADH, as NADH partially removed the effect of H₂O₂ which promotes the metHb conversion.
(3) Next, the effect of nitrite ion (NO₂⁻), which is known as oxidant for Hb, was studied. To a cuvette with an optical path length of 1 cm, 3 mL of phosphate buffered saline (PBS, GIBCO, pH 7.4), 30 μL of dispersion solution of the artificial red blood cells encapsulating NADH (Hb concentration: 9.9 g/dL or 1.5 mM) and 30 μL of NaNO₂ (1.5 mM) were mixed and the change of absorbance at 630 nm, the absorption wavelength of metHb, at 25° C. was immediately monitored for 10 minutes and for 40 minutes (FIGS. 5A and 5B). In the graphs of FIGS. 5A and 5B, the absorbance at 630 nm was put on the vertical axis and incubation time (second) was put on the horizontal axis. The two lines in FIGS. 5A and 5B represent a change over time of metHb when the dispersion solution with encapsulated NADH was supplemented with NaNO₂ (Hb-V+NADH+NaNO₂) and a change over time of metHb when the dispersion solution without encapsulated NADH was supplemented with H₂O₂ (Hb-V+NaNO₂). Increase of metHb-specific absorbance at 630 nm was remarkably suppressed when NADH was encapsulated compared with the increase when NADH was not encapsulated. It was interpreted that metHb conversion was suppressed by encapsulating NADH, as NADH partially removed the effect of NaNO₂ which promotes the metHb conversion.

### Example 4

To examine the suppression of Hb oxidation by encapsulation of NADH further in detail, similar experiments were carried out for Hb solution which was not encapsulated. The HbCO solution (42 g/dL), which did not contain any enzyme system, purified according to the method of Example 2 was diluted four fold with saline, converted to HbO₂ by light irradiation under oxygen gas flow, and used for the following experiments.

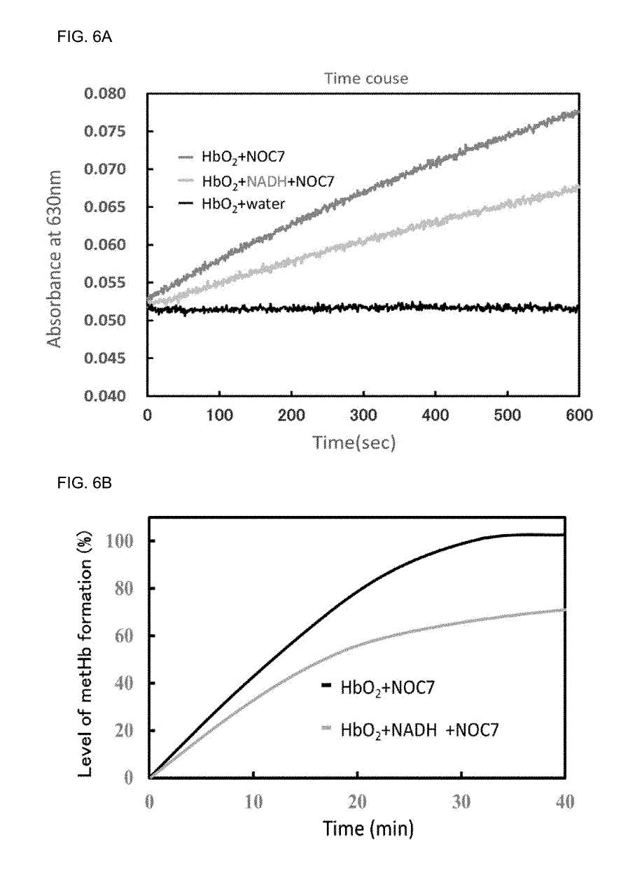

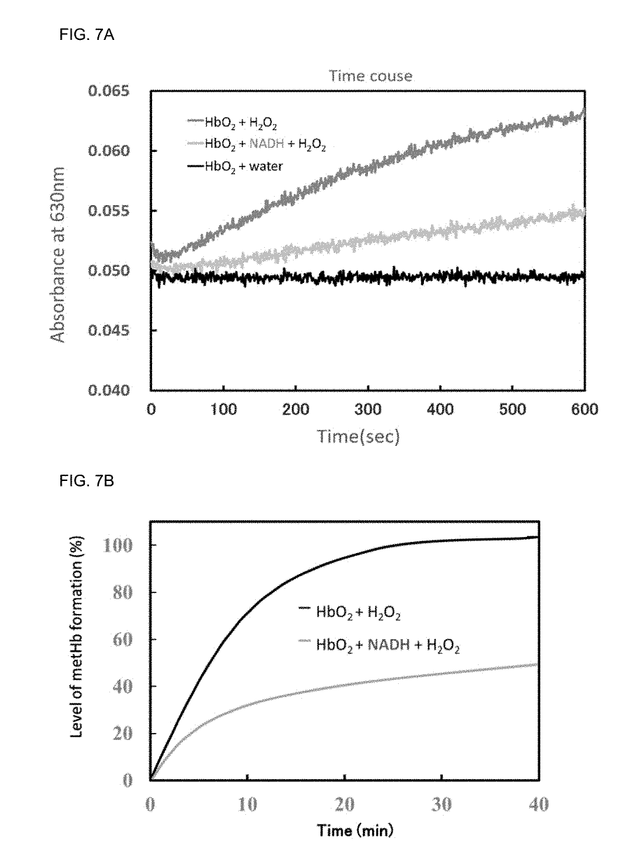

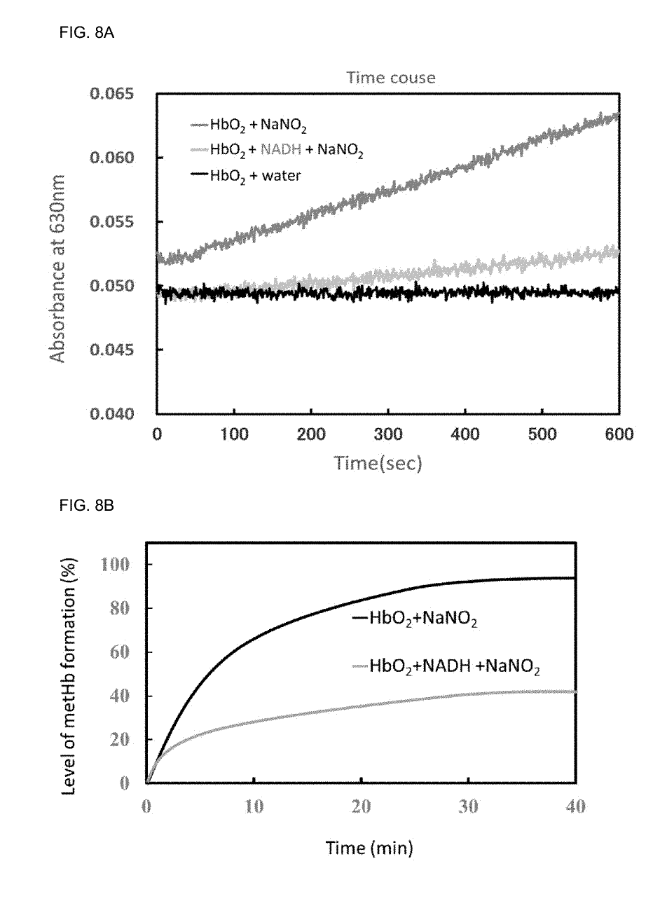

(1) First, metHb conversion supplemented with NOC7 was studied. To a cuvette with an optical path length of 1 cm, 3 mL of phosphate buffered saline, 30 μL of HbO₂ solution (1.5 mM), 30 μL of NADH solution (1.5 mM) and 30 μL of NOC (1.5 mM in PBS) were mixed and the change of absorbance at 630 nm, the absorption wavelength of metHb, at 25° C. was immediately monitored for 10 minutes and for 40 minutes (FIGS. 6A and 6B). In the graphs of FIGS. 6A and 6B, the absorbance at 630 nm was put on the vertical axis and incubation time (second) was put on the horizontal axis. The three lines in FIG. 6A represent a change over time of metHb when the solution comprising HbO₂ and NADH was supplemented with NOC7 (HbO₂+NADH+NOC7), a change over time of metHb when the solution comprising HbO₂ but not NADH was supplemented with NOC7 (HbO₂+NOC7) and a change over time of metHb when the solution comprising HbO₂ was neither supplemented with NADH nor NOC7 (HbO₂+water). The two lines in FIG. 6B represent a change over time of metHb when the solution comprising HbO₂ and NADH was supplemented with NOC7 (HbO₂+NADH+NOC7) and a change over time of metHb when the solution comprising HbO₂ but not NADH was supplemented with NOC7 (HbO₂+NOC7). Increase of metHb-specific absorbance at 630 nm was remarkably suppressed when NADH was coexisted, compared with the increase when NADH was not present.
(2) Next, the effect of supplementing with H₂O₂ was examined. To a cuvette with an optical path length of 1 cm, 3 mL of PBS, 30 μL of HbO₂ solution (1.5 mM), 30 μL of NADH solution (1.5 mM) and 30 μL of H₂O₂ (1.5 mM) were mixed and the change of absorbance at 630 nm at 25° C. was immediately monitored for 10 minutes and for 40 minutes (FIGS. 7A and 7B). In the graphs of FIGS. 7A and 7B, the absorbance at 630 nm was put on the vertical axis and incubation time (second) was put on the horizontal axis. The three lines in FIG. 7A represent a change over time of metHb when the solution comprising HbO₂ and NADH was supplemented with H₂O₂ (HbO₂+NADH+H₂O₂), a change over time of metHb when the solution comprising HbO₂ but not NADH was supplemented with H₂O₂ (HbO₂+H₂O₂) and a change over time of metHb when the solution comprising HbO₂ was neither supplemented with NADH nor NOC7 (HbO₂+water). The two lines in FIG. 7B represent a change over time of metHb when the solution comprising HbO₂ and NADH was supplemented with H₂O₂ (HbO₂+NADH+H₂O₂) and a change over time of metHb when the solution comprising HbO₂ but not NADH was supplemented with H₂O₂ (HbO₂+H₂O₂). Increase of metHb-specific absorbance at 630 nm was remarkably suppressed when NADH was coexisted compared with the increase when NADH was not present.
(3) Next, the effect of supplementing with NaNO₂ was examined. To a cuvette with an optical path length of 1 cm, 3 mL of PBS, 30 μL of HbO₂ solution (1.5 mM), 30 μL of NADH solution (1.5 mM) and 30 μL of NaNO₂ (1.5 mM in PBS) were mixed and the change of absorbance at 630 nm at 25° C. was immediately monitored for 10 minutes and for 40 minutes (FIGS. 8A and 7B). In the graphs of FIGS. 8A and 7B, the absorbance at 630 nm was put on the vertical axis and incubation time (second) was put on the horizontal axis. The three lines in FIG. 8A represent a change over time of metHb when the solution comprising HbO₂ and NADH was supplemented with NaNO₂ (HbO₂+NADH+NaNO₂), a change over time of metHb when the solution comprising HbO₂ but not NADH was supplemented with H₂O₂ (HbO₂+NaNO₂) and a change over time of metHb when the solution comprising HbO₂ was neither supplemented with NADH nor NaNO₂ (HbO₂+water). The two lines in FIG. 8B represent a change over time of metHb when the solution comprising HbO₂ and NADH was supplemented with NaNO₂ (HbO₂+NADH+NaNO₂) and a change over time of metHb when the solution comprising HbO₂ but not NADH was supplemented with NaNO₂ (HbO₂+NaNO₂). Increase of metHb-specific absorbance at 630 nm was remarkably suppressed when NADH was coexisted compared with the increase when NADH was not present.

### Example 5

To clarify the mechanism for suppressing metHb conversion by NADH, reactivity of NADH and an oxidant was examined. Hemoglobin was not added and the change of absorbance at 340 nm, the absorption wavelength of NADH, was monitored.

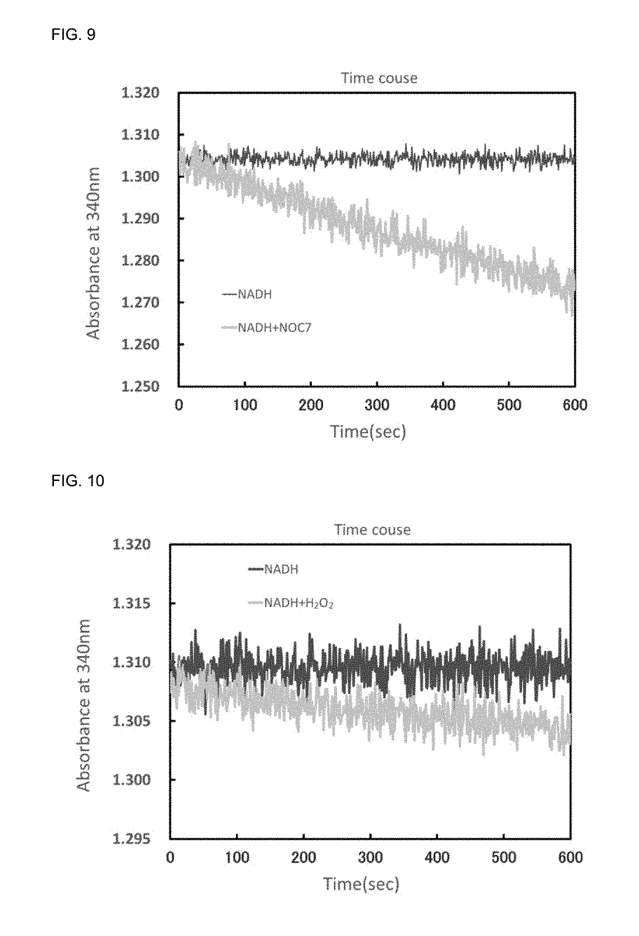

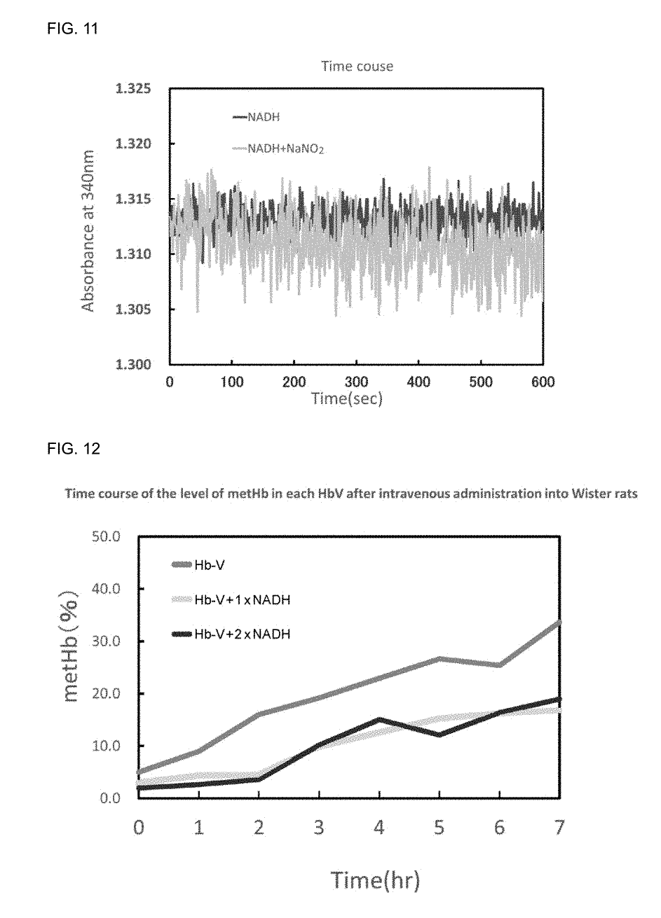

(1) First, to a cuvette with an optical path length of 1 cm, 3 mL of PBS, 0.4 mL of NADH solution (1.5 mM) and 0.4 mL of NOC7 (1.5 mM in PBS) were mixed and the change of absorbance at 340 nm at 25° C. was immediately monitored for 10 minutes (FIG. 9). In the graphs of FIG. 9, the absorbance at 340 nm was put on the vertical axis and incubation time (second) was put on the horizontal axis. The two lines in FIG. 9 represent a change over time of NADH when the NADH solution was supplemented with NOC7 (NADH+NOC7) and a change over time of metHb when the NADH solution was not supplemented with NOC (NADH). It was shown that NADH reacts with NO, as the absorbance of NADH decreased in the presence of NOC. Accordingly, the results of Examples 3 (1) and 4 (1) that promotion of metHb conversion by NOC7 was suppressed by the coexisting NADH was interpreted as caused by the activity of NADH to inactivate NO.
(2) Next, to a cuvette with an optical path length of 1 cm, 3 mL of PBS, 0.4 mL of NADH solution (1.5 mM) and 0.4 mL of H₂O₂ (1.5 mM in PBS) were mixed and the change of absorbance at 340 nm at 25° C. was immediately monitored for 10 minutes (FIG. 10). In the graphs of FIG. 10, the absorbance at 340 nm was put on the vertical axis and incubation time (second) was put on the horizontal axis. The two lines in FIG. 10 represent a change over time of NADH when the NADH solution was supplemented with H₂O₂ (NADH+H₂O₂) and a change over time of metHb when the NADH solution was not supplemented with H₂O₂ (NADH). It was shown that NADH reacts with H₂O₂, as the absorbance of NADH decreased in the presence of H₂O₂. Accordingly, the results of Examples 3 (2) and 4 (2) that promotion of metHb conversion by H₂O₂ was suppressed by the coexisting NADH was interpreted as caused by the activity of NADH to inactivate NO. In Example 3 (2), it was postulated that one reason why NADH suppresses metHb conversion is because NADH eliminated H₂O₂ which was produced by disproportion of O₂⁻., generated by the autoxidation of HbO₂. Comparing with the degree of change in the reaction to suppress the conversion of metHb in Example 4 (2), however, the reactivity of NADH and H₂O₂ is not so high. Thus, it is likely that this hypothesis alone cannot fully explain the suppression of metHb conversion by NADH. An alternative hypothesis is proposed that there is a mechanism to suppress metHb conversion by promotion of H₂O₂ inactivation (via catalase activity) due to the coexistence of Hb and NADH.
(3) Next, to a cuvette with an optical path length of 1 cm, 3 mL of PBS, 0.4 mL of NADH solution (1.5 mM) and 0.4 mL of NaNO₂ (1.5 mM in PBS) were mixed and the change of absorbance at 340 nm at 25° C. was immediately monitored for 10 minutes (FIG. 11). In the graphs of FIG. 11, the absorbance at 340 nm was put on the vertical axis and incubation time (second) was put on the horizontal axis. The two lines in FIG. 11 represent a change over time of NADH when the NADH solution was supplemented with NaNO₂ (NADH+NaNO₂) and a change over time of metHb when the NADH solution was not supplemented with NaNO₂ (NADH). It appeared that the absorbance of NADH very slightly decreased in the presence of NaNO₂. The degree of the change is very small, however, compared with the decrease when NOC7 or H₂O₂ was supplemented. This clearly demonstrates that the reactivity of NADH and NaNO₂ is low. Accordingly, it was interpreted that the results of Examples 3 (39 and 4 (3) that the coexistence of NADH suppresses promotion of metHb conversion by NaNO₂ were not caused by the action of NADH directly inactivating NaNO₂, but other action. Specifically, NADH was considered to eliminate NO and H₂O₂ generated by the reaction of NaNO₂ and HbO₂.

### Example 6

To the HbCO solution purified and concentrated in Example 2, the identical molar amount to Hb of pyridoxal 5′-phosphate (PLP, Aldrich) was added as allosteric factor. Then, powder of NADH (Orientalbio Co., Ltd.) was added to the HbCO solution with the molar ratio to Hb of 1.0 or 2.0 and resolved by gentle agitation. Powder of mixed lipid (DPPC/cholesterol/DHSG/DSPE-PEG₅₀₀₀) was added to the thick HbCO solution containing NADH and PLP. The Hb was encapsulated by kneading with the planetary movement as the principle (International Publication WO2012/137834). The dense slurry obtained by the kneading was supplemented with saline appropriately and dispersed by a further gentle kneading. The artificial red blood cells were precipitated by ultracentrifuge (50,000×g, 1 hour). The supernatant of the Hb solution which had not been encapsulated was removed. The precipitate was dispersed with saline and applied to an eggplant flask connected to a rotary evaporator. The rotary evaporator was started with a blow of oxygen passed through a gas washing bottle and irradiation of sodium lamp from outside of the vessel, to convert HbCO to HbO₂. Finally, the Hb concentration was adjusted to 10 g/dL and subjected for animal administration tests.

Wistar rats (male, weight: 350-450 g) were subjected to inhalation anesthesia with isoflurane (1-2%, FiO₂=20%) and polyethylene catheters were inserted into the femoral artery and the femoral vein. The artificial red blood cell formulation was administered intravenously. Administered were three kinds of artificial red blood cells comprising NADH whose molar ratio to Hb was 0, 1.0 and 2.0. Their dose was set at 10 mL/kg body weight. About 100 μL of blood was collected over time and applied to glass capillary for hematocrit measurement, separated by centrifuge to obtain plasma layer with dispersed artificial red blood cells in the supernatant. This was transferred to a cuvette with Thunberg tube, diluted with phosphate buffered saline (pH 7.4), sealed with a rubber plug, and conduct nitrogen bubbling by inserting a needle, to completely exclude oxygen in the solution and to make a two-component system of deoxyHb and metHb. The metHb conversion percentage was determined from the ratio of absorbance at wavelengths of maximum absorption (430 nm and 405 nm, respectively). The results are shown in FIG. 12. In the graphs of FIG. 12, the metHb conversion percentage (%) was put on the vertical axis and incubation time was put on the horizontal axis. The three lines in FIG. 12 represent a change over time of the metHb conversion percentage of the artificial red blood cells whose molar ratio of NADH to Hb was 2.0 (Hb-V+2×NADH), a change over time of the metHb conversion percentage of the artificial red blood cells whose molar ratio of NADH to Hb was 1.0 (Hb-V+1×NADH) and a change over time of the metHb conversion percentage of the artificial red blood cells which did not contain NADH (Hb-V). As illustrated in FIG. 12, systems with encapsulated NADH exert effect to suppress the metHb conversion as low as half of the control.

### Example 7

As for the pseudo activity of SOD and CAT in Example 1 (Table 1), experiments were carried out with NADPH instead of NADH. In all experiments, the results similar to those with NADH were obtained. In Examples 4 and 5, experiments were carried out with NADPH instead of NADH and the results similar to those with NADH were obtained in all experiments. It was found that the reactivity of NADH and NADPH are almost identical, as their structures are similar. It was interpreted that the effect of NADPH to suppress metHb conversion was inferior to that of NADH in the experiment to coexist with HbO₂ at 37° C. for 26 hours, because NADPH is less chemically stable than NADH (Wu, J. T., Wu, L. H., and Knight, J. A. (1986) Stability of NADPH: effect of various on the kinetics of degradation. Clin. Chem. 32, 314-319).

### Example 8

Wistar rats (male and body weight 280-350 g) were used. The rats were subjected to anesthesia similarly to Example 6 and catheters were inserted into the blood vessels. The total volume of circulating blood of the rats was estimated to be 56 mL per kg body weight. 28 mL per kg body weight of blood, corresponding to the half of the estimated total volume, was exsanguinated from the rats at the rate of 1 mL/min. 15 minutes later, the rats were administered with the same volume as the exsanguinated blood of a solution of Hb-V comprising NADH supplemented with the albumin preparation (“Benesis”, 25% albumin from donated blood for iv injection, 5 g/20 mL, Japanese Blood Products Organization (ALB)) at a volume ratio (ALB:Hb-V=1.4:8.6). Aliquots of about 100 μL were collected once every other hour immediately after the administration of the solution according to the above-mentioned protocol, and the metHb conversion percentage of the Hb-V was determined. The rats were euthanized by exsanguination from abdominal aorta, following the seventh blood collection.

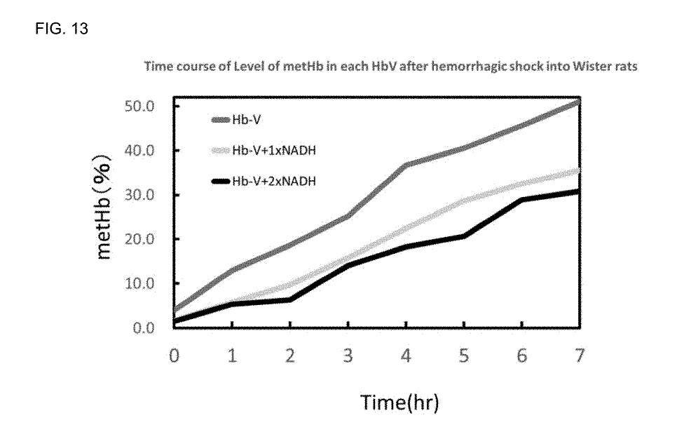

When the rats which lost 50% of the volume of circulating blood and were in a state of hemorrhagic shock were resuscitated by administrating Hb-V, the metHb conversion rate was faster than the Hb-V administered to the normal rats. The Hb-V with NADH suppressed the metHb conversion almost by half compared with the Hb-V without NADH (FIG. 13). Hb-V comprising two-fold higher dose of NADH tended to suppress the metHb conversion more effectively. After the administration, no difference of appearance was observed between rats which received Hb-V with and without NADH.

1-18. (canceled)

19. An artificial red blood cell comprising:
a capsule comprising an aqueous solution containing hemoglobin (Hb) and NADH and/or NADPH,
wherein the aqueous solution and the capsule are substantially free of enzyme activity for reducing methemoglobin (metHb).

20. The artificial red blood cell according to claim 19, wherein the 50% metHb conversion time is 72 hours or more.

21. The artificial red blood cell according to claim 19,
wherein the concentration of Hb is 10 to 45 g/dL (1.6 to 7.0 mM), and
wherein the molar concentration of NADH and/or NADPH is 0.5 to 10 times higher than the molar concentration of Hb.

22. The artificial red blood cell according to claim 19, further comprising a second aqueous solution containing pyridoxal 5′-phosphate at a molar concentration that is 0.5-3 times higher than the molar concentration of the hemoglobin in the second aqueous solution.

23. The artificial red blood cell according to claim 19, wherein the capsule comprises a liposome, a polymersome, or a thin film of polymer.

24. The artificial red blood cell according to claim 23, wherein the liposome consists of:
1,2-dipalmitoyl-sn-glycero-3-phosphatidylcholine (DPPC),
cholesterol,
1,5-O-dihexadecyl-N-succinyl-L-glutamate (DHSG), and
1,2-distearoyl-sn-glycero-3-phosphatidylethanolamine-N-PEG5000 (DSPE-PEG5000).

25. A pharmaceutical preparation for blood surrogate, comprising the artificial red blood cell according to claim 19.

26. The pharmaceutical preparation for blood surrogate according to claim 25,
wherein the artificial red blood cell is dispersed in an aqueous carrier solution that comprises at least one chemical compound selected from the group consisting of electrolytes, saccharides, amino acids, colloids, NADH, and NADPH.

27. An agent for eliminating at least one substance selected from the group consisting of NO, H₂O₂, and O₂⁻., said agent comprising
a capsule containing an aqueous solution of NADH and/or NADPH,
wherein the capsule and the aqueous solution are substantially free of enzyme activity to reduce methemoglobin (metHb), and
wherein the capsule and the aqueous solution do not comprise methylene blue.

28. A method for producing an artificial red blood cell, comprising the steps of:
(a) substantially eliminating enzyme activity to reduce methemoglobin (metHb) in a first aqueous solution comprising hemoglobin (Hb),
(b) resolving NADH and/or NADPH in the first aqueous solution and preparing a second aqueous solution comprising Hb and NADH and/or NADPH, wherein the Hb is substantially free of enzyme activity for reducing metHb,
(c) encapsulating the second aqueous solution in a capsule to obtain an artificial red blood cell consisting of the second aqueous solution and the capsule.

29. The method for producing an artificial red blood cell according to claim 28, wherein step (a) comprises heating the first aqueous solution at 60 to 65° C. for 1 to 12 hours.

30. The method for producing an artificial red blood cell according to claim 28,
wherein the Hb concentration in the second aqueous solution is 10 to 45 g/dL (1.6 to 7.0 mM), and the molar concentration of NADH and/or NADPH in the aqueous solution is 0.5 to 10 times higher than the molar concentration of the Hb.

31. The method for producing an artificial red blood cell according to claim 28, wherein in step (b), the second aqueous solution comprises pyridoxal 5′-phosphate at a molar concentration that is 0.5-3 times higher than the molar concentration of the Hb.

32. The method for producing an artificial red blood cell according to claim 31, wherein step (b) comprises resolving pyridoxal 5′-phosphate in the first aqueous solution or the second aqueous solution.

33. The method for producing an artificial red blood cell according to claim 28, wherein the capsule comprises a liposome, a polymersome, or a thin film of polymer.

34. The method for producing an artificial red blood cell according to claim 33, wherein the liposome consists of:
1,2-dipalmitoyl-sn-glycero-3-phosphatidylcholine (DPPC),
cholesterol,
1,5-O-dihexadecyl-N-succinyl-L-glutamate (DHSG), and
1,2-distearoyl-sn-glycero-3-phosphatidylethanolamine-N-PEG5000 (DSPE-PEG5000).

35. A method for treating sepsis, treating ingestion of a large amount of nitrite salt, and/or preventing methemoglobinemia during NO inhalation therapy in a subject, which comprises administering to the subject an artificial red blood cell according to claim 19.

36. The method according to claim 35, wherein the artificial red blood cell is administered in the form of a pharmaceutical preparation.

37. The method according to claim 36, wherein the pharmaceutical preparation comprises an aqueous solution, and at least one chemical compound selected from the group consisting of electrolytes, saccharides, amino acids, colloids, NADH, and NADPH.
